(19) United States
(12) Patent Application Publication
(10) Pub. No.: US 2023/0201172 A1
(43) Pub. Date: Jun. 29, 2023

(54) METHODS OF TREATING CFTR RELATED DISEASES AND DISORDERS

(71) Applicant: ARIEL PRECISION MEDICINE, INC., Pittsburgh, PA (US)

(72) Inventors: David WHITCOMB, Pittsburgh, PA (US); Mark HAUPT, Winnetka, IL (US)

(21) Appl. No.: 17/927,272

(22) PCT Filed: May 27, 2021
(86) PCT No.: PCT/US2021/034555

§ 371 Date: Nov. 22, 2022

Related U.S. Application Data
(60) Provisional application No. 63/030,618, filed on May 27, 2020.

(51) Int. Cl. A61K 31/437; A61P 1/18

(52) U.S. Cl. CPC A61K 31/437; A61P 1/18

(57) ABSTRACT

Compositions and methods that can be used to treat recurrent acute pancreatitis or a disease with disordered cystic fibrosis transmembrane conductance regulator (CFTR) function are described herein. For example, pharmaceutical compositions containing a compound of Formula I or a pharmaceutically acceptable salt thereof may be used to treat recurrent acute pancreatitis.

### CROSS-REFERENCE TO RELATED APPLICATIONS

This application claims the benefit of priority to U.S. Provisional Application No. 63/030,618 entitled “Methods of Treating CFTR Related Diseases and Disorders,” filed May 27, 2020, which is incorporated herein by reference in its entirety.

### BACKGROUND

Mutations in the CFTR gene result in CFTR dysfunction. Two severe mutations, or sequence variants, are associated with cystic fibrosis (CF) as a recessive genetic disorder. CF symptoms, such as progressive obstructive airways and eventual pancreatic insufficiency, result from deleterious variants in both copies of the CFTR gene. While only about 1 in approximately every 3000 newborns is diagnosed with CF, approximately 1 in 29 people in the US are carriers of pathogenic CFTR alleles (Cystic Fibrosis Foundation). Additionally, the effects of mutations in CFTR are not limited to cystic fibrosis. CFTR codes for a cAMP/PKA-dependent, ATP-dependent, membrane chloride ion channel, and known as CFTR (cystic fibrosis transmembrane conductance regulator). CFTR is generally found in the apical membranes of fluid-secreting or absorbing epithelial cells. There are currently over 2000 known sequence variants affecting CFTR protein, with many of these giving rise to disease phenotypes. Variants in the CFTR protein sequence result in different levels of severity of channel dysfunction with Classes I-III and VII (no protein expression) being severe and Classes IV-VI being less severe with residual channel function. As an example, around 75% of CF patients have one or two alleles that contain the class II ΔF508 mutation (or F508del) in which a codon (three nucleotides) has been lost, leading to a missing phenylalanine at position 508 in the protein. This altered protein fails to be trafficked to the correct location in the cell and is generally destroyed by the proteasome resulting in the loss of overall CFTR function. The small amount that does reach the correct location functions poorly (Cuthbert A W, British Journal of Pharmacology, 163(1), 173-183, 2011). While the CFTR protein functions primarily as a chloride channel, it has many other roles, including inhibition of sodium transport through the epithelial sodium channel, regulation of the outwardly rectifying chloride channel, ATP channels, intracellular vesicle transport, and inhibition of endogenous calcium-activated chloride channels. CFTR can also function as a bicarbonate channel. Currently, there are four FDA approved therapies that specifically target CFTR. All therapies are manufactured and marketed by Vertex Pharmaceuticals: ivacaftor (CFTR potentiator), lumacaftor/ivacaftor (combination CFTR corrector and potentiator), tezacaftor/ivacaftor (combination CFTR corrector and potentiator), and elexacaftor/tezacaftor/ivacaftor (combination CFTR corrector, potentiator and enhancer). In CF, severe CF-causing variants (Class I-III, VII) in both CFTR alleles lead to severe dysfunction or loss of the CFTR protein, which in turn reduces chloride- and/or bicarbonate-mediated fluid secretion leading to dysfunction of multiple organs that utilize CFTR for fluid secretion or absorption and subsequent pathology and organ failure. Patients with CF typically develop chronic pancreatitis with exocrine pancreatic insufficiency, sinopulmonary disorders with loss of lung function and chronic sinusitis, gastrointestinal dysfunction (for example, distal intestinal obstructive syndrome), CF-related diabetes mellitus, liver injury and reproductive disorders. CF is a multi-system disease. Diagnosis of CF requires the combination of clinical signs and symptoms, evidence of CFTR dysfunction documented by sweat chloride testing and genotyping of CFTR with identification of two pathogenic sequence variants. However, there are many complex disorders and diseases that do not meet diagnostic criteria for CF, yet are associated with mild CFTR variants or with a single CFTR variant in combination with other factors. These disorders may affect one or more than one organ or system. Patients with single organ manifestations of CFTR dysfunction do not have CF but may be classified as CFTR-Related disorder or a CF related condition if it affects an organ commonly affected in CF. These conditions/disorders include, but are not limited to, the following: pancreatitis, sinusitis, neonatal jaundice, constipation, chronic bronchitis, failure to thrive, intestinal malabsorption, pancreatic cancer and other pancreatic conditions (Miller, A. et. al. Proc. Natl. Acad. Sci. USA. 2020 Jan. 21; 117(3):1621-1627). These other chronic diseases or disorders are diagnosed by clinical or pathologic criteria without knowledge of their relationship to genetic variations in CFTR and therefore patients are not efficiently treated.

Precision medicine can be used to identify patients with signs and symptoms or early biomarkers of dysfunction in organs or tissues that utilize CFTR for one or more functions, actions or effects who have an underlying dysfunction that can be improved or corrected using a CFTR modulator and which patients have a different cause of dysfunction that is unlikely to respond to a CFTR modulator.

Cystic fibrosis patients with comorbid pancreatic diseases have been successfully treated in clinical trials with VX-770 (ivacaftor). These patients demonstrated a decreased recurrence rate of pancreatitis (Carrion, A., Borowitz, D. S., Freedman, S. D., et. al. Reduction of Recurrence Risk of Pancreatitis in Cystic Fibrosis with Ivacaftor: Case Series. J. Pediatr. Gastroenterol. Nutr. 2018; 66:451-454). While treatment using CFTR modulators in patients who have both cystic fibrosis and pancreatitis is promising, there are significant challenges in treating patients without cystic fibrosis, but do have other CFTR related disease or disorders. In large part this is because pancreatitis may be caused by a variety of underlying etiologies, only some of which will benefit from a CFTR modulating therapeutic.

The difficulty in treating patients without cystic fibrosis is also due to the difficulty of identifying the role of CFTR dysfunction without the presence of the hallmark symptoms of CF, such as early onset pulmonary disease. Additionally, each CFTR variant can manifest in a unique presentation of symptoms and responds to some therapeutics better than others. The ability to determine whether signs and symptoms of disease are in any way related to dysfunction of CFTR requires consideration of multiple factors using a precision medicine approach before treatment for CFTR dysfunction is to be prescribed. In the case of pancreatic diseases, a subset of patients have symptoms of disease related to CFTR dysfunction in pancreatic duct cells while other patients have identical or similar signs and symptoms of disease related to dysfunction of the acinar cells or other causes of pancreatitis within or outside of the pancreas (Whitcomb D C, North American Pancreatitis Study G. Pancreatitis: TIGAR-O Version 2 Risk/Etiology Checklist With Topic Reviews, Updates, and Use Primers. Clin Transl Gastroenterol. 2019; 10(6):e00027. PMID:31166201). The signs and symptoms may include unexplained abdominal pain, elevation of pancreatic digestive enzymes such as pancreatic lipase or pancreatic amylase in the blood, reduced pancreatic fluid secretion, reduced pancreatic fluid pH, reduction in pancreatic digestive enzyme production or secretion, pancreatic edema, changes in pancreatic morphology on abdominal imaging techniques, pancreatic fibrosis or scaring, one or more episodes of acute pancreatitis or combinations of clinical and pathologic features of chronic pancreatitis. Optimal treatment requires the selection of one or more agents or strategies to detect the specific causes of one or more signs of symptoms of an acute, recurrent, chronic and/or progressive disease related to CFTR dysfunction, before or after a defined disease is diagnosed. The condition of recurrent acute pancreatitis is a primary illustration of mechanism-specific treatment, with similar strategies being needed for identification of dysfunction or a disorder in other cells that utilize CFTR that have more than one mechanism of disease and where a subset of patients will benefit from modulating CFTR function while other patients with a similar clinical presentation will not benefit. Therefore, while CFTR modulators may maximize overall CFTR function, there remain no known effective treatments for the subset of patients with pancreatic disorders such as recurrent acute pancreatitis or dysfunction of other organs or tissue related to CFTR dysfunction outside of the diagnosis of cystic fibrosis.

### SUMMARY

In one embodiment, the present disclosure relates to a method of treating recurrent acute pancreatitis in a subject, the method comprising administering to the subject in need thereof a therapeutically effective amount of a pharmaceutical composition containing compound of a formula I:

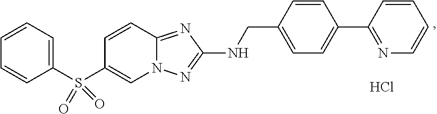 [SMILES: O=S(=O)(c1ccccc1)c1ccc2nc(NCc3ccc(-c4ccccn4)cc3)nn2c1 ; Cl]

or a pharmaceutically acceptable salt thereof. The formula of the present disclosure has a chemical name of: 6-(phenylsulfonyl)-N-(4-(pyridin-2-yl)benzyl)-[1,2,4]triazolo[1,5-a]pyridin-2-amine hydrochloride and molecular formula of C₂₄H₂₀ClN₅O₂S. The shown compound formula I is the hydrochloride salt, but the methods and compositions described herein can alternatively use the free base (C₂₄H₁₉N₅O₂S; without HCl) or other pharmaceutically acceptable salts such as mesylate, hydrobromide, acetate, fumarate, and so on.

In a another embodiment, the present disclosure relates to a method of treating a disease with disordered CFTR function, the method comprising administering to the subject in need thereof of a therapeutically effective amount of a pharmaceutical composition containing a compound of a formula:

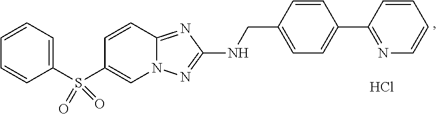 [SMILES: O=S(=O)(c1ccccc1)c1ccc2nc(NCc3ccc(-c4ccccn4)cc3)nn2c1 ; Cl]

or a pharmaceutically acceptable salt thereof. The shown compound formula is the hydrochloride salt, but the methods and compositions described herein can alternatively use the free base (without HCl) or other pharmaceutically acceptable salts such as mesylate, hydrobromide, acetate, fumarate, and so on.

### BRIEF DESCRIPTION OF THE DRAWINGS

The accompanying drawings, which are incorporated in and form a part of the specification, illustrate the embodiments of the invention and together with the written description, serve to explain the principles, characteristics, and features of the invention. In the drawings:

FIG. 1 shows the Ussing dose-response curves for potentiation of F508del-CFTR current following acute addition of ivacaftor and X0176. The x-axis is potentiator concentration in micromolar, and the y-axis is relative CFTR activity. The round symbols correspond to Ivacaftor and the square symbols correspond to the compound of Formula I (X0176), hydrochloride salt.

FIG. 2 shows F508del-CFTR dependent current in CF-hBE cells after acute or chronic potentiation. The solid bars represent the acute potentiator, and the striped bars represent the chronic potentiator. The y-axis is normalized F508del-CFTR dependent response.

### DEFINITIONS

As used herein, the term “CFTR gene” refers to the native sequence CFTR as disclosed, for example, by the National Center for Biotechnology Information https://www.nbci.nlm.nib.gov/gene/1080, including variants thereof and DNA sequence variants affecting gene expression. CFTR (italics) refers to the gene encoding the CFTR protein product and the DNA sequences within and outside of the protein coding region that regulate the genes; the CFTR locus and distant elements affecting gene expression (expression quantitative trait loci, eQTL).

As used herein, the term “CFTR modulator” refers to a class of pharmaceutical agents that act to facilitate the transport of chloride and bicarbonate ions through the ion channel of the CFTR protein. A CFTR modulator can be a CFTR corrector, a CFTR potentiator, or a CFTR enhancer.

As used herein, the term “pharmaceutical agent” or “compound” refers to a chemical entity or biological product, or combination of chemical entities or biological products, administered to a person to treat or prevent or control a disease or condition. The chemical entity or biological product is preferably, but not necessarily a low molecular weight compound, but may also be a larger compound, for example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

As used herein, the term “genotype” in the context of this invention refers to the particular allelic form of a gene, which can be defined by the particular nucleotide(s) present in a nucleic acid sequence at a particular site(s) or that regulate the expression of the gene alone or as part of a haplotype (variants in cis). The term “genotype” may also include a combination of sequence variants that may be on the alternate allele or individually or as a haplotype, or affect gene expression from outside of well-defined haplotypes and are defined as being in trans.

As used herein, the terms “variant form of a gene”, “form of a gene”, or “allele” refer to one specific form of a gene in a population, the specific form differing from other forms of the same gene in the sequence of at least one, and frequently more than one, variant sites within the sequence of the gene. The sequences at these variant sites that differ between different alleles of the gene are termed “gene sequence variances” or “variances” or “variants”. Other terms known in the art to be equivalent include mutation and polymorphism and may involve sequence deletions, insertions or copy number variants of some or all portions of the gene including regulatory elements. In preferred aspects of this invention, the variances are selected from the group consisting of the variances listed in the variance tables herein. The term variant may also be used to define an allele or haplotype and may be used to include other variants that are on the same haplotype or that are in linkage disequilibrium.

As used herein, “CFTR risk variant” refers to a variant of CFTR that confers an increased risk of CFTR related disease or disorder as compared to the risk of the disease or disorder in a subject without a CFTR variant.

As used herein, “recurrent acute pancreatitis” or “acute recurrent pancreatitis” refer to a group of disorders in which a subset of cases are associated with insufficient CFTR activity and may result in development of recurrent or persistent signs, symptoms or biomarkers of pancreatic dysfunction. Recurrent acute pancreatitis is a clinical entity characterized by episodes of acute pancreatitis which occurs on more than one occasion.

As used herein, a “tissue sample” refers to a sample of tissue or fluid isolated from an individual, including but not limited to, for example, blood, plasma, serum, tumor biopsy, mucosal swabs, hair, skin, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In a preferred embodiment, the sample is from cheek epithelial cells.

As used herein, “patient history information” refers to a survey completed by either the patient or on behalf of the patient or by the physician with data pertaining to environmental factors, personal history, medical history, surgical history, lifestyle, medications and family history.

As used herein, “precision medicine” is medical care designed to optimize efficiency or therapeutic benefit for particular groups of patients, especially by using genetic or molecular profiling. Precisions medicine refers to a methodology used to distinguish between two or more disorders with similar signs and symptoms and to inform management of the disease, disorder, and/or symptoms.

As used herein, the term “about” when immediately preceding a numerical value means a range of plus or minus 10% of that value, for example, “about 50” means 45 to 55, “about 25,000” means 22,500 to 27,500, etc., unless the context of the disclosure indicates otherwise, or is inconsistent with such an interpretation.

As used herein, the terms “administer,” “administering” or “administration” as used herein refer to directly administering a compound or a composition to a subject.

As used herein, the term “effective amount” refers to an amount that results in measurable inhibition of at least one symptom or parameter of a specific disorder or pathological process. As used herein the term “therapeutically effective amount” of compositions of the application is an amount, which confers a therapeutic effect on the treated subject, at a reasonable benefit/risk ratio applicable to any medical treatment. The therapeutic effect may be objective (that is, measurable by some test or marker) or subjective (that is, subject gives an indication of or feels an effect or physician observes a change).

As used herein the term “immediate release” refers to pharmaceutical compositions that release the active ingredient within a short period of time.

As used herein the term “modified release” refers to pharmaceutical compositions that does not otherwise release the active ingredient immediately, for example it may release the active ingredient at a sustained or controlled rate over an extended period of time, or may release the active ingredient after a lag time after administration, or may be used optionally in combination with an immediate release composition. Modified release includes extended release, sustained release and delayed release. The term “extended release” or “sustained release” as used herein is a dosage form that makes a drug available over an extended period of time after administration. The term “delayed release” as used herein is a dosage form that releases a drug at a time other than immediately upon administration.

The term “preventing” may be taken to mean to prevent a specific disorder, disease or condition and/or prevent the reoccurrence of a specific disorder, disease or condition.

As used herein, the term “prognosis” means the probable course and outcome of a disease, especially of the chances of recovery.

As used herein the terms “treat”, “treatment”, “treated”, or “treating” refer to both therapeutic treatment and prophylactic or preventative measures, wherein the object is to protect against (partially or wholly) or slow down (for example, lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this application, beneficial or desired clinical results include, but are not limited to, alleviation of symptoms; diminishment of the extent or vigor or rate of development of the condition, disorder or disease; stabilization (that is, not worsening) of the state of the condition, disorder or disease; delay in onset or slowing of the progression of the condition, disorder or disease; amelioration of the condition, disorder or disease state; and remission or recurrence (whether partial or total), whether or not it translates to immediate lessening of actual clinical symptoms, or enhancement or improvement of the condition, disorder or disease; preventing spread of the condition, disorder or disease state. Treatment seeks to elicit a clinically significant response without excessive levels of side effects. The term “unit dosage form” refers to physically discrete units suitable as a unitary dosage for human subjects and other animals, each unit containing a predetermined quantity of active material calculated to produce the desired therapeutic effect, in association with a suitable pharmaceutical excipient.

The term “disease” as used herein is intended to be generally synonymous, and is used interchangeably with, the terms “disorder,” “dysfunction,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a medical condition or disorder described in the present disclosure. Such administration encompasses co-administration of these therapeutic agents in a substantially simultaneous manner, such as in a single capsule, or dosage presentation, having a fixed ratio of active ingredients or in multiple, separate capsules for each active ingredient. In addition, such administration also encompasses use of each type of therapeutic agent in a sequential manner in the same patient, with delivery of the individual therapeutics separated by 1-24 hours, 1-7 days, or 1 or more weeks. In either case, the treatment regimen will provide beneficial effects of the drug combination in treating the conditions or disorders described herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

### DETAILED DESCRIPTION

This disclosure is not limited to the particular systems, devices and methods described, as these may vary. The terminology used in the description is for the purpose of describing the particular versions or embodiments only, and is not intended to limit the scope.

Various methods are described herein for the treatment of recurrent acute pancreatitis or a disease with disordered CFTR function. The methods include administration of at least one pharmaceutical composition to the subject. The treatment can reduce or eliminate the harmful symptoms of the disordered CFTR function.

In one embodiment, the present invention describes a method of treating recurrent acute pancreatitis in a subject, the method comprising administering to the subject in need thereof a pharmaceutical composition containing a compound of Formula I:

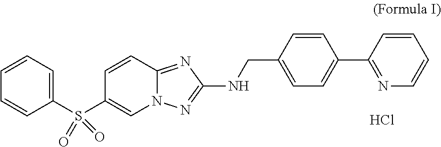 [SMILES: O=S(=O)(c1ccccc1)c1ccc2nc(NCc3ccc(-c4ccccn4)cc3)nn2c1 ; Cl]

or a pharmaceutically acceptable salt thereof. The shown compound formula I is the hydrochloride salt, but the methods and compositions described herein can alternatively use the free base (without HCl) or other pharmaceutically acceptable salts such as mesylate, hydrobromide, acetate, fumarate, and so on.

In an embodiment, the present invention discloses a method of treating a disease with disordered CFTR function, the method comprising administering to the subject in need thereof a pharmaceutical composition containing a compound of Formula I or a pharmaceutically acceptable salt thereof. The shown compound formula I is the hydrochloride salt, but the methods and compositions described herein can alternatively use the free base (without HCl) or other pharmaceutically acceptable salts such as mesylate, hydrobromide, acetate, fumarate, and so on.

Pharmaceutical Compositions

The pharmaceutical composition can comprise the CFTR potentiator of Formula I or a pharmaceutically acceptable salt thereof. The shown compound formula I is the hydrochloride salt, but the methods and compositions described herein can alternatively use the free base (without HCl) or other pharmaceutically acceptable salts such as mesylate, hydrobromide, acetate, fumarate, and so on. Methods for its preparation are shown in U.S. Pat. Nos. 9,790,215, 9,783,529, 10,370,366, 8,937,178, 9,682,969, 10,280,160, 9,855,249, 10,072,017, 9,944,603, 9,771,327, 9,573,948, 9,926,315, and 10,472,357 and PCT publications: WO2014/160478, WO2014/160440, WO2016/054560, WO2016/057522, WO2017/117239, which are incorporated herein by reference. Formula I can be present in the pharmaceutical composition at generally any effective amount or effective concentration. Different pharmaceutical forms may have different amounts or concentrations of Formula I. Example amounts include at least about 0.01 mg. Other example ranges are about 500 mg to about 5000 mg. Specific examples of ranges include about 0.01 mg to about 5000 mg, about 1 mg to about 400 mg, or about 10 mg to about 300 mg. Specific examples of amounts include about 0.01 mg, about 0.1 mg, about 1 mg, about 10 mg, about 20 mg, about, 30 mg, about 40 mg, about 50 mg, about 60 mg, about 70 mg, about, 80 mg, about 90 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, about 4000 mg, about 4100 mg, about 4200 mg, about 4300 mg, about 4400 mg, about 4500 mg, about 4600 mg, about 4700 mg, about 4800 mg, about 4900 mg, about 5000 mg, and ranges between any two of these values.

While not wishing to be bound by theory, Applicant currently believes that CFTR potentiators act to increase the function of the normal CFTR allele to overcome the diminished function attributable to the damaged CFTR variant. In one embodiment, the subject with recurrent acute pancreatitis experiences dysfunction in CFTR protein in the pancreatic ducts. With normal CFTR alleles, the pancreas produces pancreatic digestive enzymes in the acinar cells that are secreted into the pancreatic ducts. The ducts are lined by duct cells that contain CFTR which is used to transport chloride and bicarbonate ions into the duct. The chloride and bicarbonate ions are followed by sodium and water to flush the pancreatic zymogens (pre-activated digestive enzymes) out of the pancreas and into the duodenum where they are activated and digest food. If CFTR protein function is diminished the digestive enzymes are retained in the duct. When they become activated, they injure the pancreas and trigger acute pancreatitis. Increasing CFTR function to a level needed to flush the pancreatic duct will prevent the retention of pancreatic zymogens in the ducts and reduce the rate of recurrent acute pancreatitis.

In some examples, the pharmaceutical composition can further comprise one or more active compounds. This additional active compound or compounds can be used as part of a combination therapy approach. Examples of additional CFTR modulators and include: ABBV-2222 (formerly GLPG2222), ABBV-2737 (formerly GLPG2737), ABBV-2451 (formerly GLPG2451) ABBV-974 (formerly GLPG1837), ABBV-3067 (Formerly GLP3067), ABBV-191, ABBV-3221 (AbbVie), GLPG2851, GLPG1837, GLPG2451, GLPG3067 (Galapagos; Mechelen, Belgium); Riociguat (Bayer; Leverkusen, Germany); FDL-169 (Flatley Discovery Lab; Charlestown, Mass., USA); QBW251 (Novartis; Basel, Switzerland); VX-371/P-1037 (Parion Sciences; Durham, N.C., USA); PYR-41, CP-628006 (Pfizer; New York, N.Y., USA); PTI-428, PTI-801, PTI-808 (Proteostasis Therapeutics; Boston, Mass., USA, now Yumanity), NU001, NU002 (Traffick Therapeutics, Montreal, Quebec, Canada), VX-661, VX-152, VX-440, VX-445, VX-659 (Vertex Pharmaceuticals; Boston, Mass., USA).

In another embodiment, the pharmaceutical composition may be administered alone or in combination with one or more additional pharmaceutical agents to treat pancreatitis such as administering normal saline solution, administering dextrose solution, administering Ringer's lactate solution, administering albumin, administering plasma, or administering a solution of electrolytes.

In some examples, the pharmaceutical composition further comprises one or more pharmaceutically acceptable excipients. Examples of pharmaceutically acceptable excipients that may be present in the composition include but are not limited to fillers/vehicles, solvents/co-solvents, preservatives, antioxidants, suspending agents, surfactants, antifoaming agents, buffering agents, chelating agents, sweeteners, flavoring agents, binders, extenders, disintegrants, diluents, lubricants, fillers, wetting agents, glidants, and combinations thereof.

In some examples, the pharmaceutic composition can further comprise one or more exemplary fillers. Examples of exemplary fillers include cellulose and cellulose derivatives such as microcrystalline cellulose; starches such as dry starch, hydrolyzed starch, and starch derivatives such as corn starch; cyclodextrin; sugars such as powdered sugar and sugar alcohols such as lactose, mannitol, sucrose and sorbitol; inorganic fillers such as aluminum hydroxide gel, precipitated calcium carbonate, carbonate, magnesium aluminometasilicate, dibasic calcium phosphate; and sodium chloride, silicon dioxide, titanium dioxide, titanium oxide, dicalcium phosphate dihydrate, calcium sulfate, alumina, kaolin, talc, or combinations thereof. Fillers may be present in the composition from about 20 wt % to about 65 wt %, about 20 wt % to about 50 wt %, about 20 wt % to about 40 wt %, about 45 wt % to about 65 wt %, about 50 wt % to about 65 wt %, or about 55 wt % to about 65 wt % of the total weight of the composition, or any value between these ranges.

In some examples, the pharmaceutical composition further comprises one or more disintegrants. Examples of disintegrants include starches, alginic acid, crosslinked polymers such as crosslinked polyvinylpyrrolidone, croscarmellose sodium, potassium starch glycolate, sodium starch glycolate, clays, celluloses, starches, gums, or combinations thereof. Disintegrants may be present in the composition from about 1 wt % to about 10 wt %, about 1 wt % to about 9 wt %, about 1 wt % to about 8 wt %, about 1 wt % to about 7 wt %, about 1 wt % to about 6 wt %, or about 1 wt % to about 5 wt % of the total weight of the composition, or any value between these ranges.

In some examples, the pharmaceutical composition further comprises one or more binders, including but not limited to celluloses such as hydroxypropylcellulose, methyl cellulose, and hydroxypropylmethylcellulose; starches such as corn starch, pregelatinized starch, and hydroxypropyl starch; waxes and natural and synthetic gums such as acacia, tragacanth, sodium alginate; synthetic polymers such as polymethacrylates and polyvinylpyrrolidone; and povidone, dextrin, pullulane, agar, gelatin, tragacanth, macrogol, or combinations thereof. Binders may be present in the composition from about 0.5 wt % to about 5 wt %, about 0.5 wt % to about 4 wt %, about 0.5 wt % to about 3 wt %, about 0.5 wt % to about 2 wt %, or about 0.5 wt % to about 1 wt % of the total weight of the composition, or any value between these ranges.

In some examples, the pharmaceutical composition further comprises one or more wetting agents, including but not limited to oleic acid, glyceryl monostearate, sorbitan mono-oleate, sorbitan monolaurate, triethanolamine oleate, polyoxyethylene sorbitan mono-oleate, polyoxyethylene sorbitan monolaurate, sodium oleate, sodium lauryl sulfate, poloxamers, poloxamer 188, polyoxyethylene ethers, polyoxyethylene sorbitan fatty acid esters, polyoxyethylene fatty acid esters, polyethylene glycol fatty acid esters, polyoxyethylene hardened castor oil, polyoxyethylene alkyl ethers, polysorbates, cetyl alcohol, glycerol fatty acid esters (for example, triacetin, glycerol monostearate, etc.), polyoxymethylene stearate, sodium lauryl sulfate, sorbitan fatty acid esters, sucrose fatty acid esters, benzalkonium chloride, polyethoxylated castor oil, and combinations thereof. Wetting agents may be present in the composition from about 0.1 wt % to about 1 wt %, about 0.1 wt % to about 2 wt %, about 0.1 wt % to about 3 wt %, about 0.1 wt % to about 4 wt %, or about 0.1 wt % to about 5 wt % of the total weight of the composition, or any value between these ranges.

In some examples, the pharmaceutical composition further comprises one or more lubricants, including but not limited to stearic acid, magnesium stearate, calcium hydroxide, talc, corn starch, sodium stearyl fumarate, alkali-metal and alkaline earth metal salts, waxes, boric acid, sodium benzoate, sodium acetate, sodium chloride, leucine, polyethylene glycol (PEG), a methoxypolyethylene glycol, propylene glycol, sodium oleate, glyceryl behenate, glyceryl palmitostearate, glyceryl benzoate, magnesium lauryl sulfate, sodium lauryl sulfate, and combinations thereof. Lubricants may be present in the composition from about 0.1 wt % to about 5 wt %, about 0.1 wt % to about 4 wt %, about 0.1 wt % to about 3 wt %, about 0.1 wt % to about 2 wt %, or about 0.1 wt % to about 1 wt % of the total weight of the composition, or any value between these ranges.

In some examples, the pharmaceutical composition further comprises one or more glidants, including but not limited to colloidal silicon dioxide, talc, sodium lauryl sulfate, native starch, and combinations thereof. Glidants may be present in the composition from about 0.05 wt % to about 1 wt %, about 0.05 wt % to about 0.9 wt %, about 0.05 wt % to about 0.8 wt %, about 0.05 wt % to about 0.5 wt %, or about 0.05 wt % to about 0.1 wt % of the total weight of the composition, or any value between these ranges.

In some examples, the pharmaceutical composition is a tablet and further comprises a top coat, such as hydroxypropyl-methylcellulose coating or polyvinyl alcohol coating, and are available under the trade name Opadry, such as Opadry White, Opadry II (Opadry is a registered trademark of BPSI Holdings LLC, Wilmington, Del., USA). Top coats may be present in the composition from about 1 wt % to about 10 wt %, about 1 wt % to about 9 wt %, about 1 wt % to about 8 wt %, about 1 wt % to about 7 wt %, about 1 wt % to about 6 wt %, or about 1 wt % to about 5 wt % of the total weight of the composition, or any value between these ranges.

In some examples, the pharmaceutical composition can further comprise one or more preservative agents. Examples of preservative agents include sodium benzoate, paraoxybenzoic acid esters, methyl, ethyl, butyl, and propyl parabens, chlorobutanol, benzyl alcohol, phenylethylalcohol, dehydroacetic acid, sorbic acid, benzalkonium chloride (BKC), benzethonium chloride, phenol, phenylmercuric nitrate, thimerosal, or combinations thereof. Preservative agents can be included in the liquid dosage form. The preservative agents can be in an amount sufficient to extend the shelf-life or storage stability, or both, of the liquid dosage form. Preservatives may be present in the composition from about 0.05 wt % to about 1 wt %, about 0.05 wt % to about 0.9 wt %, about 0.05 wt % to about 0.8 wt %, about 0.05 wt % to about 0.5 wt %, or about 0.05 wt % to about 0.1 wt % of the total weight of the composition, or any value between these ranges.

In some examples, the pharmaceutical composition can further comprise one or more flavoring agents. Examples of flavoring agents include synthetic flavor oils and flavoring aromatics and/or natural oils, extracts from plants leaves, flowers, fruits, and so forth and the like or any combinations thereof. Additional examples include cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil and the like or any combinations thereof. Also useful as flavors are vanilla, citrus oil, including lemon, orange, grape, lime and grapefruit, and fruit essences, including apple, banana, pear, peach, strawberry, raspberry, cherry, plum, pineapple, apricot, strawberry flavor, tutti-fruity flavor, mint flavor, or any combinations thereof. Flavoring agents may be present in the composition from about 0.1 wt % to about 5 wt %, about 0.1 wt % to about 4 wt %, about 0.1 wt % to about 3 wt %, about 0.1 wt % to about 2 wt %, or about 0.1 wt % to about 1 wt % of the total weight of the composition, or any value between these ranges.

Physical Form of the Pharmaceutical Composition

The pharmaceutical composition can generally be in any physical form suitable for use in treating a subject. These forms can be referred to as a unit dosage form, such as an individual pill or tablet. In some examples, the pharmaceutical compositions can be formulated as tablets, capsules, granules, powders, liquids, suspensions, gels, syrups, slurries, suppositories, patches, nasal sprays, aerosols, injectables, implantable sustained-release formulations, or mucoadherent films. In some examples, the pharmaceutical composition may be formed as a tablet, a bi-layer tablet, a capsule, a multiparticulate, a drug coated sphere, a matrix tablet, or a multicore tablet. A physical form can be selected according to the desired method of treatment.

Pharmaceutical composition can be manufactured by various conventional methods such as conventional mixing, dissolving, granulating, dragee-making, levigating, emulsifying, encapsulating, entrapping, or lyophilizing processes. Pharmaceutical compositions can be formulated in a conventional manner using one or more physiologically acceptable carriers, diluents, excipients or auxiliaries that facilitate processing of the active agent into preparations that can be used pharmaceutically. Proper formulation can be selected upon the route of administration chosen.

For topical administration the pharmaceutical composition described herein may be formulated as solutions, gels, ointments, creams, suspensions, and the like as are well-known in the art. Systemic compositions include, but are not limited to, those designed for administration by injection, for example, subcutaneous, intravenous injection (IV), intramuscular injection (IM), intrathecal injection (IT), intraperitoneal injection (IP), as well as those designed for transdermal, subcutaneous, transmucosal oral, or pulmonary administration. For injection, the pharmaceutical compositions can be formulated in aqueous solutions, preferably in physiologically compatible buffers such as Hanks solution, Ringer's solution, or physiological saline buffer and/or in certain emulsion formulations. The solution can contain one or more formulatory agents such as suspending, stabilizing and/or dispersing agents. In certain examples the pharmaceutical compositions can be provided in powder form for constitution with a suitable vehicle, for example, sterile pyrogen-free water, before use. For transmucosal administration, one or more penetrants appropriate to the barrier to be permeated can be used in the formulation. Such penetrants are generally known in the art.

For oral administration, the pharmaceutical composition can combine Formula I or Formula I with another pharmaceutical agent with one or more pharmaceutically acceptable carriers well known in the art. Such carriers facilitate formulation as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like can be added. For buccal administration, the compositions may take the form of tablets, lozenges, etc. formulated in conventional manner.

For administration by inhalation, the pharmaceutical composition can be delivered in the form of an aerosol spray from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, for example, gelatin for use in an inhaler or insufflator may be formulated containing a powder mix of the compound and a suitable powder base such as lactose or starch.

In some examples, the pharmaceutical compositions are immediate release pharmaceutical compositions, modified release pharmaceutical compositions, or a combination thereof. In some examples, the immediate release pharmaceutical composition releases Formula I or a pharmaceutically acceptable salt thereof within a short period of time after administration, typically less than about 4 hours, less than about 3.5 hours, less than about 3 hours, less than about 2.5 hours, less than about 2 hours, less than about 90 minutes, less than about 60 minutes, less than about 45 minutes, less than about 30 minutes, less than about 20 minutes, or less than about 10 minutes.

In some examples, the modified release composition may release the Formula I or a pharmaceutically acceptable salt thereof at a sustained or controlled rate over an extended period of time, or may release it after a lag time after administration. For example, it may be released from the composition 4 hours after administration, 8 hours after administration, 12 hours after administration, 16 hours after administration, or 24 hours after administration. Modified release compositions include extended release, sustained release and delayed release compositions. In some examples, the modified release compositions may release about 10% in about 2 hours, about 20% in 2 hours, about 40% in about 2 hours, about 50% in about 2 hours, about 10% in about 3 hours, about 20% in 3 hours, about 40% in about 3 hours, about 50% in about 3 hours, about 10% in about 4 hours, about 20% in 4 hours, about 40% in about 4 hours, about 50% in about 4 hours, about 10% in about 6 hours, about 20% in 6 hours, about 40% in about 6 hours, or about 50% in about 6 hours.

In some examples, modified release compositions may comprise a matrix selected from microcrystalline cellulose, sodium carboxymethylcellulose, hydroxyalkylcelluloses such as hydroxy propyl methylcellulose and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acetate trimellitate, polyvinyl acetate phthalate, polyalkylmethacrylates, polyvinyl acetate and mixtures thereof.

The modified release compositions can also be formulated as a depot preparation. Such long acting formulations can be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Methods of Treatment

The compounds and pharmaceutical compositions described herein may be administered at therapeutically effective dosage levels to treat the recited conditions, disorders, and diseases.

The compounds and pharmaceutical compositions described herein may be administered at prophylactically effective dosage levels to mitigate or prevent the recited conditions, disorders, and diseases.

Administration may be performed by generally any method. Example delivery methods of administering include topical delivery, subcutaneous delivery, intravenous injection (IV) delivery, intramuscular injection (IM) delivery, intrathecal injection (IT) delivery, intraperitoneal injection (IP) delivery, transdermal delivery, subcutaneous delivery, oral delivery, transmucosal oral delivery, pulmonary delivery, inhalation delivery, intranasal delivery, buccal delivery, rectal delivery, vaginal delivery, and combinations thereof. In some examples, the administering comprises oral delivery.

The daily dose of Formula I or a pharmaceutically acceptable salt thereof can generally be any effective amount or dosage. For example, the therapeutically effective amount may include about 0.01 mg to about 5000 mg, about 1 mg to about 400 mg, or about 10 mg to about 300 mg. Specific examples of therapeutically effective amounts include about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 2000 mg, about 3000 mg, about 4000 mg, about 5000 mg, and ranges between any two of these values. For example, the daily dosage can be about 100 mg/kg/day, about 0.01 mg/kg/day, about 0.1 mg/kg/day to about 5 g/kg/day, about 0.01-100 mg/kg/day, about 0.01-50 mg/kg/day, about 0.01-25 mg/kg/day, about 0.01-10 mg/kg/day, or about 0.01-1 mg/kg/day. Specific examples include about 0.01 mg/kg/day, about 0.05 mg/kg/day, about 0.1 mg/kg/day, about 0.5 mg/kg/day, about 1 mg/kg/day, about 2 mg/kg/day, about 5 mg/kg/day, about 10 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day, about 200 mg/kg/day, about 300 mg/kg/day, about 400 mg/kg/day, about 500 mg/kg/day, about 600 mg/kg/day, about 700 mg/kg/day, about 800 mg/kg/day, about 900 mg/kg/day, about 1000 mg/kg/day, about 2000 mg/kg/day, about 3000 mg/kg/day, about 4000 mg/kg/day, about 5000 mg/kg/day, or ranges between any two of these values. When administered in two or more daily doses, the amount in each dose can be added together to yield a total daily dose.

Use of the described methods and pharmaceutical compositions can result in a reduction or elimination of disease, symptom, or other undesired property in a subject relative to a control population (for example, without treatment by the described methods and materials). The reduction can generally be reduced by any amount. For example, the reduction can be at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, and in an ideal situation, about 100% reduction (complete elimination of disease, symptom, virus concentration, or other undesired property).

In some examples, the methods disclosed herein to treat recurrent acute pancreatitis have the following primary outcome measures: reduction in the episodes of abdominal pain, or the severity of abdominal pain, or the frequency of episodes of acute pancreatitis. In some examples, the methods disclosed herein to treat recurrent acute pancreatitis have the following secondary outcome measures: reduction in the number of sick days from work or school, reduction in the use of pain medication, improvement in the quality of life, reduction in biomarkers of chronic pancreatitis. In another example a patient with recurrent acute pancreatitis that is determined to have pathogenic CFTR variants or CFTR risk variants have the following primary outcome: reduction of sweat chloride concentration of at least 5 mEq following administration of a compound as an indicator of improved patient CFTR channel function.

Methods of Potentiating CFTR Function

The compounds and pharmaceutical compositions described herein are CFTR potentiators. CFTR potentiators improve CFTR function by altering chloride channel gating to increase the open probability of the CFTR protein. CFTR modulators are sensitive to the CFTR variant of individuals and the magnitude of response to a CFTR therapeutic agent is highly correlated to the amount of residual CFTR function (Han S T, Rab A, Pellicore M J, et al. Residual function of cystic fibrosis mutants predicts response to small molecule CFTR modulators. JCI Insight. 2018; 3). For example, a method of improving the fluid secretory function of the pancreatic duct cells, acinar cells and centroacinar cells following diagnosis of acute or recurrent acute pancreatitis comprises administering to subject in need thereof a pharmaceutical composition comprising an effective amount of Formula I or a pharmaceutically acceptable salt thereof. The shown compound formula I is the hydrochloride salt, but the methods and compositions described herein can alternatively use the free base (without HCl) or other pharmaceutically acceptable salts such as mesylate, hydrobromide, acetate, fumarate, and so on.

In some examples, a method of treating chronic sinusitis is a subject with or without a history of pancreatitis comprises administering to subject in need thereof a pharmaceutical composition comprising an effective amount of Formula I or a pharmaceutically acceptable salt thereof. The shown compound formula I is the hydrochloride salt, but the methods and compositions described herein can alternatively use the free base (without HCl) or other pharmaceutically acceptable salts such as mesylate, hydrobromide, acetate, fumarate, and so on.

Subjects to be Treated

The subject can generally be any mammal. Examples of subjects include a primate, a human, a dog, a cat, a mouse, a rat, a cow, a horse, a pig, a rabbit, and a ferret. In some examples, the subject is a human. The terms “subject”, “individual”, or “patient” are used interchangeably and as used herein are intended to include human and non-human animals. Non-human animals include all vertebrates, for example, mammals and non-mammals, such as non-human primates, sheep, dogs, rats, cats, cows, horses, ferrets, chickens, amphibians, and reptiles. Examples of mammals include non-human primates, sheep, dogs, cats, cows, ferrets, and horses. In some examples, the subject is a human or humans. The methods are suitable for treating humans having a CFTR related disease.

Methods of generating a treatment recommendation for a subject diagnosed with recurrent acute pancreatitis are described, the method comprising receiving patient history information associated with the subject, receiving information on clinical signs, symptoms or biomarkers associated with pancreatic disease, receiving genetic information associated with the subject, identifying at least one genetic variance within a CFTR allele of a subject based on the received genetic information, selecting a CFTR modulator according to the identified genetic variance, the patient history information, patient biomarker information that may include laboratory tests, or imaging tests, functional tests, special tests that may include sweat chloride testing or nasal potential difference testing or other direct or indirect tests of CFTR function and providing a recommendation to treat the subject with the selected compound of Formula I and treating the subject with the selected compound of Formula I. The method of generating a treatment recommendation may be used to determine that a patient, who is suspected of having an underlying disorder of the pancreas that may lead to the diagnosis of a disease at a later time, has genetic variants that affect CFTR expression or function and may be successfully treated using the compound of Formula I treatment. In these patients, use of Formula I treatment may be beneficial to improve the symptoms, to slow the rate of disease progression, or to prevent the development of a pancreatic disease. The method may also be used to determine if the signs, symptoms or biomarkers of a pancreatic disorder or disease will be responsive to Formula I including disease related to the pancreatic duct cells, the pancreatic duct content, or pancreatic duct function, or unlikely to respond to Formula I such as diseases related to pancreatic acinar cell function such as mutations in the trypsinogen gene, PRSSI, hypercalcemia, calcium dysregulation within the acinar cell or misfolding variants in proteins synthesized by the acinar cells. The term recurrent acute pancreatitis refers to a group of disorders in which a subset of cases are associated with insufficient CFTR activity and may result in development of recurrent or persistent signs, symptoms or biomarkers of pancreatic dysfunction including elevated serum or plasma amylase, lipase, trypsin, trypsinogen or other pancreatic digestive enzymes, abdominal pain, abdominal discomfort, nausea, pancreatic inflammation that in some cases may be diagnosed as acute pancreatitis, recurrent acute pancreatitis, acute recurrent acute pancreatitis, chronic pancreatitis, pancreatitis pseudocysts, abnormal pancreatic secretion, and associated with or increase the risk of type 1 diabetes, type 2 diabetes, type 3 diabetes, CFTR dysfunction in pancreatitis, CF-related diabetes, secondary diabetes, cyst or pseudocyst of the pancreas, pancreatic calcifications, other pancreatic disorders, pancreatic pain disorders, pancreatic fibrosis, pancreatic cancer, or biliary disease.

In one embodiment, the disease with disordered CFTR function is acute pancreatitis, chronic pancreatitis, pediatric pancreatitis, pancreatic cancer, abdominal pain, or diabetes.

In one embodiment, the method further comprises determining if the patient has a comorbid condition to recurrent acute pancreatitis. In an embodiment, the comorbid condition is a sweat chloride concentration greater than or equal to 30 mmol/L. In an embodiment, the comorbid condition is a sweat chloride concentration of about 30 mmol/L to about 60 mmol/L. In another embodiment, the comorbid condition is a sweat chloride concentration equal to or greater than 60 mmol/L. In one embodiment, the comorbid condition is an intermediate sweat chloride concentration. In one embodiment, the comorbid condition is a high sweat chloride concentration. In one embodiment the method further comprises other direct or indirect tests of CFTR function which measure the effects of CFTR channel function in a biological system, in a tissue, or in a cell (either in vivo or in vitro) that are obvious to those trained in the art such as nasal potential difference measures, secretin-stimulated pancreatic function test using an intestinal or pancreatic duct fluid sampling system, or measured using imaging technologies.

In one embodiment, the method of treating a subject comprises administering to the subject who may benefit from a pharmaceutical composition containing Formula I or a pharmaceutically acceptable salt thereof wherein the subject has signs, symptoms or biomarkers of, or is diagnosed with at least one of the following conditions at a stage or mechanism predicted to improve with CFTR modulators: conditions of the liver, bile ducts, and gallbladder affecting the biliary duct system where insufficient CFTR activity may result in development of abnormal liver injury tests with increase in serum bilirubin or other abnormal liver serum enzyme levels with or without cholestasis, cholelithiasis, bile duct stones, biliary diseases, biliary acute pancreatitis, biliary cirrhosis, jaundice, neonatal jaundice chronic hepatitis, liver cirrhosis, gallbladder dysfunction (including dyskinesia) or gallstones. Sinorespiratory disorders arising from epithelial cells where insufficient CFTR activity may result in upper respiratory infection, respiratory failure, respiratory failure of a newborn, chronic sinusitis, recurrent sinusitis, nasal polyposis, pneumonia, recurrent pneumonia, chronic bronchitis, pseudomonas infections, aspergillosis associated lung disease, bronchiectasis, asthma, cough (cough variant asthma), or pulmonary fibrosis. Gastrointestinal disorders arising from epithelial cells where insufficient CFTR activity may result in salivary gland dysfunction, salivary gland stones, dry mouth, tooth decay, tooth loss, periodontitis, eosinophilic esophagitis, gastroesophageal reflux disease, Barrett's esophagus, esophageal cancer, duodenitis, duodenal ulcers, peptic ulcers, intestinal sensitivity to NSAIDS, intestinal sensitivity to aspirin, celiac disease, diarrhea, nausea, vomiting, abdominal pain, constipation, fecal impaction, intestinal obstruction, intestinal astresia, inflammatory bowel disease, malnutrition, maldigestion, pancreatic steatorrhea, failure to thrive in a child, short stature, feeding difficulties and mismanagement, cachexia and adult failure to thrive, lack of expected normal development, meconium peritonitis, meconium obstruction in fetus or newborn, stomach cancer, pancreatic cancer, gallbladder cancer, cancer in gastrointestinal organs, colon cancer. Other disorders arising directly or indirectly from epithelial cells where insufficient CFTR activity may result in nephrolithiasis, dehydration, fluid and electrolyte disorders, male infertility, low sperm count, hemoptysis or hypertrophic osteoarthropathy.

In one embodiment, the method of treating a subject comprises administering to the subject in need thereof a pharmaceutical composition containing Formula I or a pharmaceutically acceptable salt thereof, wherein the subject has a decreased pancreatic fluid secretion compared to healthy subjects and treatment with a medication to increase CFTR function may be helpful to increase pancreatic fluid secretion. Disease and disorders with decreased pancreatic fluid secretion include: pancreas divisum, pancreatic duct strictures, partial or incomplete pancreatic duct obstruction, smoking, gastric acid suppression, altered surgical anatomy such as gastric bypass, roux-en-Y, duodenectomy, surgical bypass of the duodenum, anticholinergic medications, achlorhydria, chronic gastritis, vagotomy, and gastroparesis.

In one embodiment, genetic variance in the CFTR gene is identified from tissue samples collected from cheek epithelial cells. In another embodiment, genetic variance in the CFTR gene is identified from tissue samples collected from saliva. In other embodiments, tissue sample refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent.

In one embodiment, the genetic variance is located within the coding region of CFTR. In one embodiment, the genetic variance is located within the noncoding region of CFTR. In one embodiment, the genetic variance in the CFTR gene results in a change in protein structure. In one embodiment, the genetic variance in the CFTR gene results in a change in protein expression. In one embodiment, the genetic variance in the CFTR gene results in a change in regulatory elements. The genetic variance in the CFTR gene is selected from the following mutations:

A46D, G85E, E92K, P205S, R334W, R347P, T3381, S492F, I507del, V520F, A559T, R560S, R560T, A561E, L927P, H1054D, G1061R, L1065P, R1066C, R1066H, R1066M, L1077P, H1085R, M1101K, W1282X, N1303K, E56K, G178R, S549R, S977F, F1074L, 2789+5G→A, P67L, E193K, G551D, F1052V, D1152H, 3272-26A→G, R74W, L206W, G551S, K1060T, G1244E, 3849+10kbC→T, D110E, R347H, D579G, A1067T, S1251N, D110H, R352Q, 711+3A→G, G1069R, S1255P, R117C, A455E, E831X, R1070Q, D1270N, R117H, S549N, S945L, R1070W, G1349D, G970R, R75Q, F508C, F508del, G542X, M470V, T854T, Q1463Q, P1290P, G576A, R31C, R170H, R74Q, D1270N, L997F, L967S, S1235R, 1807M, V201M, R851L, V754M, 5T-TG13, 9T, T7; TG10, D1445N, A349V, F693L, R258G, M348K, V920M, R506T, 2814insA, Q1042X, S997F, 1148T, D565G, M9521, 1506V, E528E, R170C, R24W, D170N, V5021, R668C, rs17451754.

In one embodiment, genetic variance in CFTR result in a change in CFTR gene expression compared to subjects with normal CFTR. In one embodiment, CFTR gene expression is altered in c.-234T>A, c.-1750A>G, and 5T genetic variants.

In one embodiment, CFTR variants can be classified into seven distinct classes (I-VII). In one embodiment, subjects with Class I, Class II, Class III or Class VII CFTR variants have an increased phenotypic disease severity and worse prognosis of disease as compared to other classes. In one embodiment, subjects with Class VI, Class V, or Class VI CFTR variants have residual CFTR protein function and have a better prognosis of disease. In one embodiment, subjects contain multiple classes of CFTR variants. In one embodiment, subjects may have one normal CFTR allele and a Class I, Class II, Class III, or Class VII CFTR variant, where a CFTR modulator can increase the function of the CFTR protein produced by the normal or Class IV-VI CFTR allele.

Method of Identifying Subjects for Treatment

In one embodiment, a compound of Formula I or a pharmaceutically acceptable salt thereof is selected as treatment using precision medicine, where a medical professional or an analytical platform can successfully distinguish between two or more disorders with similar signs, symptoms or biomarkers of dysfunction or disease. In one embodiment, the success of treatment with a CFTR modulator is predicted by which CFTR variants are present in the subject. In another embodiment, the success of treatment with a CFTR modulator is determined using information sources selected from the group consisting of current symptoms, subject genetic information, biomarkers, and patient history information.

In one embodiment patient history information is reported by the patient. This information includes but is not limited to: past medical history, prior or ongoing symptoms, review of systems, surgical history, current and past medications, allergies, family medical history, environmental exposure history (such as alcohol, tobacco and illicit drug use), social history (such as travel history, home environment), and laboratory history (including prior laboratory testing results, imaging results and special tests). In one embodiment a treatment recommendation for a patient is generated from the patient history information. In one embodiment patient history information is reported by the patient. In another embodiment the patient history information is reported by a guardian or caregiver. In one embodiment patient history information is reported by a physician, wherein the physician is selected from the group consisting of a primary care physician, an emergency room physician, an internal medicine physician, and a genetic counselor.

### EXAMPLES

### Example 1: Identification of a Patient Who May Benefit from a CFTR Modulator

The example patient will be a 32 year old female with a past medical history of chronic abdominal pain that started at 22 years of age. The pain will be initially diagnosed as chronic functional abdominal pain. Frequent hospitalizations will be required for pain management. There is no reported surgical history. Current symptoms will also include vertigo, migraine headaches and allergies. There will be a family history of chronic sinusitis, inflammatory bowel disease, lactose intolerance. A family history of cystic fibrosis is noted in distant cousins. There will be no reported history of tobacco, alcohol, or illicit drug use. There will be no known environmental toxin exposures. No history of recent international travel will be noted. Laboratory data will not reveal any clinically significant deviations from normal values. Abdominal imaging studies suggest pancreatic enlargement of unclear etiology. The data collected for this history will require information from multiple sources, including the patient, provider, hospital records, a genetic counseling consult. Given the nature of the symptoms and clinical history and lack of identified etiology for the pancreatic enlargement, sequencing of the CFTR gene will be indicated. DNA sequencing results will identify three variants in CFTR: p.G551D, p.T854=, p.V470M. The patient is heterozygous for all variants. The methods and systems used to diagnose the patient and identify effective treatments will be done using precision medicine.

### Example 2: Identification of CFTR Variant in a Patient with Recurrent Acute Pancreatitis

The example patient will be a 30 year old male diagnosed with idiopathic recurrent acute pancreatitis. The incident event occurs at 23 years of age. After the initial episode the patient will experience 6 episodes of acute pancreatitis, each requiring hospitalization. At the time of diagnosis, current symptoms will include abdominal pain, bloating, constipation. A history of tobacco and alcohol use (quantity not specified) will be noted. There is no history of surgical procedure. His medications will include narcotics as needed for pain relief. The family history will include pancreatitis in the patient's sister. Given the diagnosis of idiopathic recurrent acute pancreatitis sequencing of the CFTR gene will be indicated. Results of DNA sequencing will identify the patient as a heterozygous carrier of the p.G551D variant, which is sensitive to the CFTR potentiator ivacaftor. Based on the clinical history, active symptoms, genetic sequencing, use of Formula I could be indicated for this patient. After initiation of therapy a reduction in the frequency of episodes of acute pancreatitis will be noted in addition to pain relief, improved digestion symptoms, improvement in anthroprometric measurements and biomarkers of nutrition. The patient will not require further hospitalizations for pain. Opioid use will be decreased.

### Example 3: Administering Pharmaceutical Composition to Subjects

Examples of using Formula I in non-pancreatic conditions are the following:

The patient will be a 67 year old male with chronic obstructive pulmonary disease. Pulmonary function testing demonstrates reduced forced expiratory volume-1 second (FEV1). Chest imaging will demonstrate bronchiectasis and air trapping. Evaluation of his lung disease includes sequencing of the CFTR gene, which will identify the p.D1152H variant. Formula I will be prescribed and the patient will demonstrate improvement in lung function and reduction in daily symptoms, such as cough and dyspnea.

The patient will be a 28 year old female with chronic sinusitis. Prior treatments include multiple course of oral antibiotics, surgical debridement, and chronic intra-nasal steroids. Symptoms will have persisted despite these therapies. Investigation into the etiology of her sinusitis will include sequencing of the CFTR gene, which will identify the p.G551D variant. Formula I will be prescribed and the patient will demonstrate improvement in daily symptoms of sinusitis, including headaches, facial pain, and congestion.

The patient will be a 64 year old male with chronic pancreatitis and Type 3c diabetes mellitus. His glucose control will be described as brittle and will be recalcitrant to typical therapies despite reasonable adherence to prescribed therapies and lifestyle modifications. Evaluation of the etiology of his pancreatitis will include sequencing of the CFTR gene, which is identified as a risk variant in CFTR. Formula I will be prescribed and the patient will demonstrate stabilization of his blood glucose measures and reduction in his hemoglobin-A1C.

An example of use of Formula I in a non-pancreatic condition related to CFTR dysfunction is the following:

The patient will be a 27 year old female with ulcerative colitis who develops jaundice. Primary sclerosing cholangitis (PSC) will be identified as the etiology of the jaundice. During the evaluation, the patient is noted to have the p.G551D variant in the CFTR gene. Given her clinical history and laboratory findings, Formula I will be prescribed, which will improve her jaundice and slow the PSC progression.

### Example 4: Beneficial Effects of Treatment with Formula I as Compared to Patients Receiving a Placebo Control

An example of a clinical trial comparing use of Formula I to placebo is the following. The study will employ a prospective randomized placebo-controlled trial structure and administer Formula I or a pharmaceutically acceptable salt thereof as an oral agent in pill, tablet, or solution form as permitted by manufacturing needs. Formulation delivery will additionally depend on the population studied to meet developmental needs, such as liquid formulation for pediatric patients. Adult and/or pediatric patients with recurrent acute pancreatitis in whom a CFTR variant was identified with some evidence of CFTR dysfunction on sweat chloride testing will be enrolled at participating centers per typical enrollment procedures. Typical inclusion and exclusion criteria will be applied to ensure the safety of participants and to avoid bias. Participants will be randomized to Formula I or placebo. Participants will receive the agents or placebo for a set period of time to evaluate changes in clinically meaningful endpoints as well as exploratory endpoints. Formula I will be administered daily as a chronic therapy. Frequency of dosing will be, at a minimum, once daily or as frequently as required to achieve safe and likely efficacious serum levels. Specific dose and frequency will be established by typical pharmacokinetic/pharmacodynamic studies. Endpoints will include, but are not limited to, frequency of attacks of acute pancreatitis, reduction in sweat chloride values, patient reported outcome measure, and measures of nutritional status. If interim analysis demonstrated significant benefit without evidence of safety concerns, participants enrolled in the placebo arm will be crossed-over to the active agent arm. Comparisons between all measures will determine efficacy and safety. Anticipated results include decreased frequency of acute pancreatitis episodes, decreased severity of acute pancreatitis episodes, reduction in pain, improved digestive symptoms, improved patient reported outcomes and quality of life measures.

### Example 5. Potentiator Activity of X0176 in CF-hBE Cells: Acute Effects on CFTR Activity

Methods: The activity of CFTR potentiator X0176 (a compound of the present disclosure, Formula I: 6-(phenylsulfonyl)-N-(4-(pyridin-2-yl)benzyl)-[1,2,4]triazolo[1,5-a]pyridin-2-amine hydrochloride) to acutely stimulate the ion current of F508del-CFTR was measured in CF-hBE cells from three CF donors in combination with the correctors FDL169, lumacaftor, and tezacaftor; results were compared with ivacaftor.

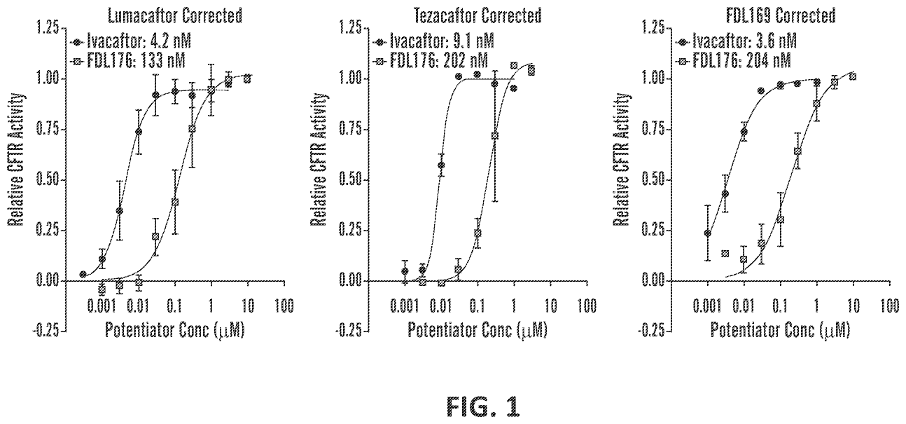

Electrophysiology assays were conducted in an Ussing chamber in which corrected cells were treated with ascending concentrations of X0176 (0.001-10 μM) or ivacaftor (0.0003-1.0 μM). Sodium channels were initially blocked with benzamil, CFTR channels were then activated with addition of forskolin, followed by acute addition (<1 hour) of increasing concentrations of X0176 or ivacaftor. At each concentration tested, the CFTR-mediated chloride current (after subtraction of the vehicle response) was normalized to the response of ivacaftor (1 μM), the reference standard, which was assigned a value of 1.0 (FIG. 1). FIG. 1 legend: Ussing assay dose-response curves for potentiation of F508del-CFTR current following acute addition of ivacaftor (0.0003-1.0 μM, black circles) and X0176 (0.001-10 μM, blue squares labeled FDL176). Cells were corrected with lumacaftor (left), tezacaftor (middle), or FDL169 (right), each at 3 μM. For each corrector treatment group, dose response data for ivacaftor and X0176 were normalized to the response following activation with 1 μM ivacaftor (with matched corrector). Error bars represent SEM. CF-hBE=cystic fibrosis primary human bronchial epithelial cells; CFTR=cystic fibrosis transmembrane conductance regulator; SEM=standard error of the mean.

Results: XO176 potency was similar across CF-hBE cells treated with all three correctors. The EC50 for lumacaftor was 0.133±0.0181 μM, for tezacaftor was 0.202±0.0446 μM and for FDL169 was 0.204±0.0244 μM. Potency of ivacaftor in corrector treated cells (EC50 range 0.0036 to 0.0091 μM), was 40- to 66-fold higher than X0176. Maximum efficacy (Emax) of X0176 in cells previously treated with the three correctors was similar to that of ivacaftor, with both X0176 and ivacaftor reaching a relative efficacy of 1.0.

Conclusions: The potency and efficacy of X0176 in combination with FDL169 were similar in all donor CF-hBE cells.

### Example 6: Potentiator Activity of XO176 in CF-hBE Cells: Chronic Effects on CFTR Activity

Methods: To investigate the effects of chronic potentiator exposure, F508del-CFTR activity was measured in an equivalent current assay in CF-hBE cells co-treated with FDL169 and XO176, lumacaftor and ivacaftor, and tezacaftor and ivacaftor for 24 hours. CFTR activity was then measured following activation with forskolin. The study was designed to compare the Emax of each corrector-potentiator pair, and maximally efficacious concentrations for the correctors (3 μM) and potentiators (3 μM for X0176; 0.1 μM for ivacaftor) were selected for evaluation. The chronic condition was compared with acute stimulation with potentiator (and simultaneous addition of forskolin) of cells previously treated for 24 hours with corrector alone.

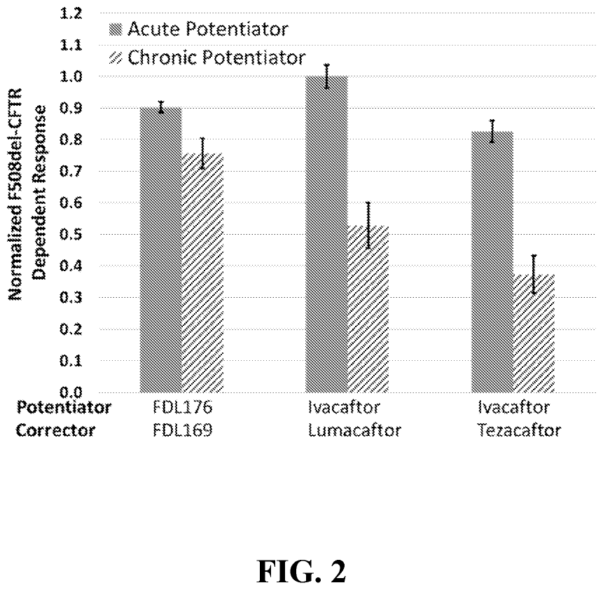

Results: The results of N=4 experiments in CF-hBEs from two donors (total of N=8 experiments) are shown in FIG. 2 (Legend: Normalized F508del-CFTR dependent current (relative response) of CF-hBE cells treated with FDL169-X0176 (presented as FDL176 in this graph), lumacaftor-ivacaftor, and tezacaftor-ivacaftor under conditions of acute (solid bars) or chronic (hashed bars) potentiator stimulation. Data were the average of N=8 experiments conducted in CF-hBE cells derived from two donors (014M and 009L). Cystic fibrosis transmembrane conductance regulator specific response was measured as the AUC of the Ieq vs time graph, measured between CFTR activation with addition of forskolin and potentiator, and CFTR inhibition with addition of CFTR-172. The AUC for the test article (AUCTA), and the reference standard lumacaftor (3 μM)+ivacaftor (0.1 μM), AUCSTD, were measured and averaged. For replicate measurements within an experiment, the average test current was normalized by dividing by the average AUCSTD to give the normalized F508del-CFTR dependent current. Differences under chronic conditions were statistically significant between FDL169-X0176 and lumacaftor-ivacaftor, and between FDL169-X0176 and tezacaftor-ivacaftor. CFTR=cystic fibrosis transmembrane conductance regulator; CF hBE=cystic fibrosis primary human bronchial epithelial cells; AUC=area under the concentration-time curve; Ieq=equivalent current; TA=test article; STD=standard). Activity of the three corrector-potentiator combinations was similar when a potentiator was added acutely to CF-hBE cells following 24 hours treatment with a corrector. Consistent with previous measurements, FDL169-X0176 maximum relative activity was approximately 0.90±0.017 vs. lumacaftor-ivacaftor (the reference standard, activity defined as 1.0), whereas tezacaftor-ivacaftor relative activity was lower, 0.83±0.034 of lumacaftor-ivacaftor.

Conclusions: Following 24 hours exposure to potentiator and corrector, the activity of FDL169-X0176 was 1.4-fold higher than lumacaftor-ivacaftor, and 2.0-fold higher than tezacaftor-ivacaftor.

### Example 7. Clinical Studies with XO176

XO176 will be further studied in patient with diseases related to CFTR dysfunction. The efficacy of XO176 will be compared to placebo in the relevant studies. Disease specific relevant outcomes will be evaluated to demonstrate the safety and efficacy of XO176. Study design will meet all relevant FDA guidance and will follow typical FDA drug development pathways. Sample sizes will be calculated to best fit the study design and to make reasonable statistical conclusions from the available clinical data. The duration of the studies will sufficient to demonstrate clinically significant outcomes as required by the FDA and as supported by the toxicology data. The following clinical outcomes may be anticipated if XO176 restores CFTR function sufficiently in the respective patient populations:

Recurrent Acute Pancreatitis: Reduction of acute pancreatitis events, pain relief, reduction in gastrointestinal symptoms, like pain, gas and bloating, improvement in quality of life.

Chronic Sinusitis: Reduction in sinusitis symptoms, resolution of sinusitis imaging findings, improvement in patient reported outcome measures.

Biliary Disease: Reduction in acute biliary events, resolution of imaging findings, improvement of quality of life, reduced disease progression.

COPD: Improvement in lung function as measured by FEV1, improved quality of life and patient reported outcomes, decrease cough.

Exocrine Pancreatic Insufficiency: Improved quality of life and patient reported outcomes, decreased nutritional failure, decreased symptoms of exocrine pancreatic insufficiency.

Dry Eye: Decreased symptoms, improved quality of life and patient reported outcomes.

### Example 8: Dose Justification

The 40 mg daily oral dose is based on the following rationale. NOAEL exposures in rats and dogs provided a 3.4- and 1.1-fold safety margin over a 50 mg human dose, respectively. The XO176 EC50 for the G551D variant, a clinically relevant CFTR variant and commonly used potentiator standard, is 2600 nM. Total plasma Cmin at steady state at the 30 mg and 50 mg dose is 2180 nM and 3170 nM, respectively. Therefore, a 40 mg human dose likely provides continued coverage of the EC50 of the G551D variant for the full dosing interval.

In the above detailed description the illustrative embodiments described in the detailed description and claims are not meant to be limiting. Other embodiments may be used, and other changes may be made, without departing from the spirit or scope of the subject matter presented herein. It will be readily understood that the aspects of the present disclosure, as generally described herein, can be arranged, substituted, combined, separated, and designed in a wide variety of different configurations, all of which are explicitly contemplated herein.

1. A method of treating recurrent acute pancreatitis in a subject, the method comprising administering to the subject in need thereof a therapeutically effective amount of a pharmaceutical composition containing a compound of Formula I: 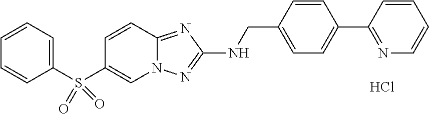 [SMILES: O=S(=O)(c1ccccc1)c1ccc2nc(NCc3ccc(-c4ccccn4)cc3)nn2c1 ; Cl]
or a pharmaceutically acceptable salt thereof.

2. The method of claim 1, wherein the pharmaceutical composition is formulated as a tablet, capsule, granule, powder, liquid, suspension, gel, syrup, slurry, suppository, patch, nasal spray, aerosol, injectable, implantable sustained-release formulation, or mucoadherent film.

3. The method of claim 1, wherein the administering comprises topical delivery, subcutaneous delivery, intravenous injection (IV) delivery, intramuscular injection (IM) delivery, intrathecal injection (IT) delivery, intraperitoneal injection (IP) delivery, transdermal delivery, subcutaneous delivery, oral delivery, transmucosal oral delivery, pulmonary delivery, inhalation delivery, intranasal delivery, buccal delivery, rectal delivery, vaginal delivery, or combinations thereof.

4. The method of claim 1, wherein the administering comprises oral delivery.

5. The method of claim 1, wherein the subject is a mammal.

6. The method of claim 1, wherein the subject is a primate, human, cat, dog, pig, cow, goat, horse, sheep, rabbit or ferret.

7. (canceled)

8. The method of claim 1, wherein the compound of Formula I is a hydrochloride salt.

9. The method of claim 1, wherein the compound of Formula I is a free base.

10. The method of claim 1, wherein the compound of Formula I is a mesylate salt, a hydrobromide salt, an acetate salt, or a fumarate salt.

11. A method of treating a disease with disordered CFTR function in a subject, the method comprising administering to the subject in need thereof a therapeutically effective amount of a pharmaceutical composition containing a compound of a formula: 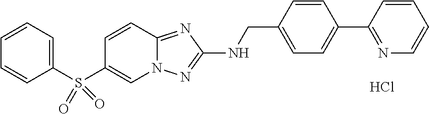 [SMILES: O=S(=O)(c1ccccc1)c1ccc2nc(NCc3ccc(-c4ccccn4)cc3)nn2c1 ; Cl]
or a pharmaceutically acceptable salt thereof.

12. The method of claim 11, wherein the disease with disordered CFTR function is acute pancreatitis, chronic pancreatitis, pediatric pancreatitis, pancreatic cancer, abdominal pain, or diabetes.

13. The method of claim 11, wherein the pharmaceutical composition is formulated as a tablet, capsule, granule, powder, liquid, suspension, gel, syrup, slurry, suppository, patch, nasal spray, aerosol, injectable, implantable sustained-release formulation, or mucoadherent film.

14. The method of claim 11, wherein the administering comprises topical delivery, subcutaneous delivery, intravenous injection (IV) delivery, intramuscular injection (IM) delivery, intrathecal injection (IT) delivery, intraperitoneal injection (IP) delivery, transdermal delivery, subcutaneous delivery, oral delivery, transmucosal oral delivery, pulmonary delivery, inhalation delivery, intranasal delivery, buccal delivery, rectal delivery, vaginal delivery, or combinations thereof.

15. The method of claim 11, wherein the administering comprises oral delivery.

16. The method of claim 11, wherein the subject is a mammal.

17. The method of claim 11, wherein the subject is a primate, cat, dog, pig, cow, goat, horse, sheep, rabbit, or ferret.

18. The method of claim 11, wherein the subject is a human.

19. The method of claim 11, wherein the compound of Formula I is a hydrochloride salt.

20. The method of claim 11, wherein the compound of Formula I is a free base.

21. The method of claim 11, wherein the compound of Formula I is a mesylate salt, a hydrobromide salt, an acetate salt, or a fumarate salt.
